SEQUENCE LISTING (27 sequences)

SEQ ID NO: 1 - protein, 12 aa, Human immunodeficiency virus
GRKKRRQRRRPQ

SEQ ID NO: 2 - protein, 16 aa, Drosophila sp.
RQIKIWFQNRRMKWKK

SEQ ID NO: 3 - protein, 9 aa, Artificial Sequence
  Description of Artificial Sequence Synthetic peptide
RRRRRRRRR

SEQ ID NO: 4 - protein, 18 aa, Mus musculus
LLIILRRRIRKQAHAHSK

SEQ ID NO: 5 - protein, 22 aa, Unknown
  Description of Unknown p14ARF peptide
MVTVLFRRLRIRRACGPPRVRV

SEQ ID NO: 6 - protein, 18 aa, Artificial Sequence
  Description of Artificial Sequence Synthetic peptide
KLALKLALKALKAALKLA

SEQ ID NO: 7 - protein, 27 aa, Artificial Sequence
  Description of Artificial Sequence Synthetic peptide
GWTLNSAGYLLGKINLKALAALAKKIL

SEQ ID NO: 8 - protein, 15 aa, Flock house virus
RRRRNRTRRNRRRVR

SEQ ID NO: 9 - protein, 21 aa, Artificial Sequence
  Description of Artificial Sequence Synthetic peptide
KETWWETWWTEWSQPKKKRKV

SEQ ID NO: 10 - protein, 18 aa, Artificial Sequence
  Description of Artificial Sequence Synthetic peptide
VRLPPPVRLPPPVRLPPP

SEQ ID NO: 11 - protein, 7 aa, Hepatitis B virus
LCLRPVG

SEQ ID NO: 12 - protein, 17 aa, Unknown
  Description of Unknown alpha-1-anti-trypsin peptide
CSIPPEVKFNKPFVYLI

SEQ ID NO: 13 - protein, 26 aa, Artificial Sequence
  Description of Artificial Sequence Synthetic peptide
GALFLGFLGAAGSTMGAWSQPKSRKV

SEQ ID NO: 14 - protein, 34 aa, Herpes simplex virus
DAATATRGRSAASRPTERPRAPARSASRPRRPVD

SEQ ID NO: 15 - protein, 7 aa, Simian virus 40
PKKKRKV

SEQ ID NO: 16 - protein, 16 aa, Unknown
  Description of Unknown Nucleoplasmin peptide
KRPAATKKAGQAKKKK

SEQ ID NO: 17 - protein, 38 aa, Unknown
  Description of Unknown hnRNP A1 peptide
NQSSNFGPMKGGNFGGRSSGPYGGGGQYFAKPRNQGGY

SEQ ID NO: 18 - protein, 11 aa, Unknown
  Description of Unknown Nuclear respiratory factor 2 peptide
EEPPAKRQCIE

SEQ ID NO: 19 - protein, 42 aa, Human immunodeficiency virus
DTWTGVEALIRILQQLLFIHFRIGCRHSRIGIIQQRRTRNGA

SEQ ID NO: 20 - protein, 12 aa, Artificial Sequence
  Description of Artificial Sequence Synthetic peptide
GGPKKKRKVGGC

SEQ ID NO: 21 - protein, 21 aa, Artificial Sequence
  Description of Artificial Sequence Synthetic peptide
LLIILRRRIRKQAHAHSKGGC

SEQ ID NO: 22 - protein, 33 aa, Artificial Sequence
  Description of Artificial Sequence Synthetic polypeptide
GGPKKKRKVGGCCGGKSHAHAQKRIRRRLIILL

SEQ ID NO: 23 - protein, 18 aa, Artificial Sequence
  Description of Artificial Sequence Synthetic peptide
LLIILRRRIRKQAHAHSK

SEQ ID NO: 24 - protein, 24 aa, Artificial Sequence
  Description of Artificial Sequence Synthetic peptide
CGGKETWWETWWTEWSQPKKKRKV

SEQ ID NO: 25 - protein, 14 aa, Artificial Sequence
  Description of Artificial Sequence Synthetic peptide
GRKKRRQRRRPPQC

SEQ ID NO: 26 - protein, 36 aa, Artificial Sequence
  Description of Artificial Sequence Synthetic polypeptide
GGPKKKRKVGGCCGGKETWWETWWTEWSQPKKKRKV

SEQ ID NO: 27 - protein, 26 aa, Artificial Sequence
  Description of Artificial Sequence Synthetic peptide
GGPKKKRKVGGCCQPPRRRQRRKKRG

(19) United States
(12) Patent Application Publication
(10) Pub. No.: US 2017/0035914 A1
(43) Pub. Date: Feb. 9, 2017

(54) FUNCTIONALIZED PEPTIDE TRANSPORTERS FOR CELLULAR UPTAKE

(71) Applicant: General Electric Company, Schenectady, NY (US)

(72) Inventors: Erik Leeming Kvam, Niskayuna, NY (US); Michael James Rishel, Saratoga Springs, NY (US); Andrew Arthur Paul Burns, Niskayuna, NY (US)

(21) Appl. No.: 14/818,372

(22) Filed: Aug. 5, 2015

(51) Int. Cl. A61K 49/00

(52) U.S. Cl. CPC A61K 49/0056

(57) ABSTRACT

Disclosed are novel cell-penetrating transporters for enhancing cellular uptake of peptide fusion proteins into live cells. The cell-penetrating transporters comprise a functionalized peptide construct comprising a cell importation peptide covalently bound to a cargo, the cell importation peptide in an unreactive monomeric form, and a pharmaceutical carrier. In certain embodiments, the cargo further comprises a nuclear localization sequence (NLS) allowing the cargo to be transported across the nuclear membrane.

### CROSS-REFERENCE TO RELATED APPLICATIONS

This application is related to U.S. patent application entitled “Methods for Enhancing Cellular Uptake of Functionalized Peptide Constructs” filed concurrently herewith under attorney docket number 280124-1; the entire disclosure is incorporated herein by reference.

### SEQUENCE LISTING

The instant application contains a Sequence Listing which has been submitted electronically in ASCII format and is hereby incorporated by reference in its entirety. Said ASCII copy, created on Jul. 23, 2015, is named 280124A-1_SL.txt and is 8,579 bytes in size.

### BACKGROUND

Recombinant proteins have been long introduced into live cells using DNA- or RNA-based techniques. For example, numerous viral-, chemical-, and physical-based methods exist to transfect recombinant nucleic acids into cells to synthesize a desired protein product that may elicit an intracellular response or may be exploited for imaging techniques. However, this fundamental practice can be indirect and inefficient in cases where the nucleotide sequence is poorly expressed and/or poorly delivered into cells. Moreover, nucleic acids may continue to propagate in cells and elicit long-term intracellular responses. A more direct method for introducing recombinant proteins into live cells is to synthesize the protein in vitro and deliver the protein directly into cells. Methods for “protein transfection” into cells can include similar viral-, chemical-, and physical-based methods. However, a preferred method is to utilize amino acid sequences that intrinsically penetrate cell membranes through passive or active mechanisms. Such amino acid sequences are referred to as cell-penetrating peptides, protein-transduction domains, and the like. These amino acid sequences may be recombinantly fused to peptide cargo to create a synthetic protein that penetrates live cells and subsequently elicit an intracellular response. Furthermore, the synthetic protein fusion may be further labeled with chemical, pharmaceutical, or fluorescent groups to transport these agents directly into cells. This fact is considered a distinct advantage over DNA- or RNA-based recombinant techniques that generally may not code for chemical, pharmaceutical, or fluorescent cargos or payloads.

While numerous cell-penetrating peptide fusion constructs exist in the art, a key limitation of any fusion constructs is that the efficiency of their cell uptake may be low or unpredictable. One common practice to resolve this issue and enhance protein transfection efficiency is to increase the concentration of the peptide fusion exposed to cells. However, this practice is costly and potentially wasteful because higher amounts of in vitro-synthesized recombinant proteins are required upfront, and subsequent chemical-conjugation reactions with chemical, pharmaceutical, or fluorescent groups may not be cost- or labor-effective at such higher scale. Furthermore, applying higher amounts of recombinant protein to cells may elicit undesired secondary or off-target effects. Thus, methods for transfecting protein into live cells at higher efficiency and lower consumption of recombinant protein are needed in the art. Furthermore, there is a need for more biologically inert labeling approaches that can enable cell imaging analysis without propagatible or long-term deleterious effects.

Recently, colorimetric and fluorescent imaging has expanded to live-cell labeling capitalizing on dyes that can pass through the cellular and nuclear membranes. The most convenient method for segmentation of cells grown in culture is by fluorescent labeling of nuclei. Because the nucleus is generally surrounded by cytoplasm on all sides, nuclei appear as individual points, rather than the continuously labeled field seen in cytoplasmic or cell surface staining. While a range of nuclear labels exist today, they generally act through direct binding to DNA, which may perturb cellular processes, and thus there exist very few fluorescent agents that are well-tolerated by cells.

A family of bis-benzimide dyes has been developed by Hoechst A.G. (Frankfurt am Main, Germany) (Hoechst dyes 33258, 33342 and 34580) with fluorescence emission wavelengths in the 400-500 nm range (with excitation wavelengths in the 300-400 nm wavelength range). These bis-benzimide dyes have been used for nuclear labeling in live cells, but in general the imaging times have been relatively short, and the cells have often been immortalized cell lines rather than primary or stem-cell derived cells, which tend to be much more fragile. A number of deleterious effects of labeling with Hoechst dyes have been recognized in the literature including inhibition of chromosome condensation, and inhibition of DNA synthesis at high concentrations (>20 mM). Further, the low wavelength excitation makes these Hoechst dyes prone to generation of radical species which can cause phototoxic effects in cells over long imaging times. An alternative family of live-cell fluorophores is the DRAQ™ dyes (BioStatus LTD, Leicestershire, UK), a family of anthraquinone derivatives (DRAQ5 and DRAQ7) which exhibit fluorescence in roughly the Cy5 and Cy7 wavelength bands (650-700 nm and 750-800 nm), respectively. These dyes exhibit lower phototoxicity, but remain DNA-binding probes. Though no structures have been published in the RSCB Protein Database (PDB) (Research Collaboratory for Structural Bioinfomatics), the consensus is that these probes bind to DNA double helices, but without the sequence specificity (e.g., A-T rich regions bound by Hoechst dyes). Further, labeling of proliferative cells shows an arrest of cell development in the G2 phase prior to mitosis as well as decreases in transcription rates (approximately 50%), polymerase activity and a variety of chromatin-associated processes in a dose-dependent manner.

Thus, in specific fields of study, there is a need for more biologically inert nuclear labeling approaches which can enable nuclear imaging and segmentation without the deleterious effects of directly binding to DNA.

Furthermore, the mechanism used to transport the nuclear labelling fluorophores, may be expanded to include a general method of delivering materials to the interior of a cell for variety of applications including: therapeutics (e.g., molecular agents for modification of cell function or initiation of cell death), biopharmaceutical and industrial (gene delivery for generation of desired proteins) and diagnostic (delivery of sub-cellular contrast agents or markers) wherein delivery of constructs within a cell requires crossing the cell membrane with minimal disruption.

### BRIEF DESCRIPTION

Disclosed herein are novel methods for enhancing the cell uptake of cell-penetrating peptide fusion proteins into live cells

In some embodiments a cell-penetrating transporter for enhancing the delivery of imaging and therapeutic agents to the interior of a cell is described. The agent comprises a functionalized peptide construct having cell importation peptide covalently bound to a cargo, the cell importation peptide in an unreactive monomeric form, and a pharmaceutical carrier.

In certain embodiment, the covalent bond between the cell importation peptide and the cargo is cleavable to allow releasing the cargo in the interior of the cell.

In yet another embodiment, transporter further provides for transporting the cargo into the interior of the cell and across the nuclear membrane, whereby the cargo comprises a nuclear localization sequence (NLS).

### DESCRIPTION OF THE FIGURES

FIG. 1 is a flowchart showing process steps for enhancing delivering a cargo to an interior of a viable cell using an aqueous solution of the CPP-cargo construct such that the CPP is present both in the functionalized peptide construct as well as a monomeric form.

FIG. 2 is a flowchart showing process steps for delivering a cargo into the cell nucleus.

FIG. 3 is a flowchart showing detailed process steps for preparing, incubating, and imaging live cells in the presence of an aqueous solution comprising a CPP-cargo construct and monomeric CPP peptide.

FIG. 4 depicts a comparison of representative cell penetrating fusion proteins and uses for labeling nuclei in live cells. A fusion protein comprising a pVEC peptide was found to deliver the NLS-FAM cargo moiety into various cell types with high efficiency.

FIG. 5a is representative HPLC data showing a first preparation which was found to contain significant carryover of unconjugated pVEC peptide shown UV absorbance at 190 nm.

FIG. 5b is corresponding MS extracted ion of FIG. 5a.

FIG. 5c is representative HPLC data showing of the second preparation showing only trace carryover of unconjugated NLS-FAM peptide.

FIG. 5d is corresponding MS extracted ion of FIG. 5c.

FIG. 6 shows fluorescent images of different cell-uptake efficiency between the first and second preparation of pVEC-NLS-FAM fusion protein.

FIG. 7 depicts fluorescent images that illustrate the effects of using different amounts of monomeric pVEC together with purified pVEC-NLS-FAM fusion protein to improve cell uptake efficiency.

FIG. 8 are fluorescent micrographs showing homologous and heterologous testing of pVEC as an adjuvant to peptide-mediated uptake using alternative cell penetrating peptides (PEP1 and TAT1).

FIG. 9 are micrographs illustrating the effect of monomer pVEC addition on the uptake of monomeric SV40 NLS peptide containing a reactive 3-nitro-2-pyridinesulfenyl cysteine residue.

### DETAILED DESCRIPTION

The singular forms “a” “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least each numerical parameter should at least be construed in light of the number of reported significant digits and by applying ordinary rounding techniques.

The term “cargo” or “payload” refers to a biological agent or reagent that is transported across the cellular membrane, for example that of a mammalian tissue sample. The cellular membrane is a lipid bilayer and as such the cargo is delivered by attachment or fusion to a transporter capable of penetrating the membrane.

The term “construct” refers to a synthesis or formation of a more complex substance. As used here it refers to the synthesized compound comprising a cargo component and a transporter component.

A construct may exhibit enhanced uptake if it if it associates more frequently with, more rapidly with, for a longer duration with, or with greater affinity to, or if it is adsorbed or absorbed more, or accumulates more in a biological sample compared to a control construct or a construct used as a comparative sample under the same conditions.

The term “tissue” or “viable cellular” sample refers to a sample obtained from a biological subject, including samples of biological tissue or fluid origin obtained in vivo or in vitro whose viability is retained. Such samples can be, but are not limited to, organs, tissues, fractions, cells isolated from mammals including, humans and cell organelles. Biological samples also may include sections of the biological sample including tissues (e.g., sectional portions of an organ or tissue). Biological samples may also include tissues cultures grown from a harvested tissue. Biological samples may comprise proteins, carbohydrates or nucleic acids.

The term “pharmaceutical carrier” may be any compatible, non-toxic substance suitable for delivery of the construct to the tissue sample to have an effective residence time for action of the construct or to provide a convenient manner of release. The pharmaceutical carrier may include sterile water, salts, alcohol, fats, waxes and inert solids for solubilization and stabilization. Solubilization strategies may include but are not limited to pH adjustments, salt formation, formation of ionizable compounds, use of co-solvents, complexation, surfactants and micelles, emulsions and micro-emulsions. The pharmaceutical carrier may include, but is not limited to, a solubilizer, detergent, buffer solution, stabilizers, and preservatives. Examples of these include but are not limited to, HCl, citric acid, DMSO, propylene glycol, ethanol PEG 300, cyclodextrans, and salts of citrate, acetate, phosphate, carbonate or tris(hydroxymethyl)aminomethane. In certain embodiments, the pharmaceutical carrier is preferably an aqueous carrier. A variety of aqueous sterile carriers may be used, for example, water, buffered water, 0.4% saline, or 0.3% glycine.

“Cell-penetrating peptides” (CPPs), refers to peptides which can translocate through the cell membrane without the need for a receptor. CPPs are peptides which can translocate through the cell membrane without the need for a receptor and are typically peptides of fewer than 30 residues derived from natural or unnatural proteins or chimeric sequences. Often the CPPs are peptides of 10 to 30 residues, and have cationic or amphipathic sequences for efficient translocation. There are several physical mechanisms by which cell-penetrating peptides can gain entry into cells, and these mechanisms can change based on the moieties to which the peptides are attached, For example HIV-1 TAT48-60 peptide, can when bound to small molecules, undergo translocation across the membrane, whereas when bound to e.g., particulate cargo, TAT mediates uptake via endocytosis into vesicles. Other CPPs can form multimeric complexes at the cell surface and form a pore through which material is transported.

Nuclear localization sequences” (NLS) are peptide sequences that can translocate across a cell nucleus and thus act as a nuclear transporter. Typically, the NLS peptide sequence consists of one or more short sequences of positively charged lysines or arginines exposed on the protein surface. In certain instances the peptide sequence may have dual functionality as a CPP and NLS.

Table 1. is a listing of exemplary peptide sequences with their function and source.

TABLE 1
Functional peptide sequences

| Type | Name(s) | Sequence | Source |
|---|---|---|---|
| CPP | TAT | GRKKRRQRRRPQ | HIV-1 TAT₄₈₋₆₀ |
| CPP | Penetratin | RQIKIWFQNRRMKWKK | Drosophila |
| | | | Antennapedia |
| | | | homeodomain |
| CPP | Arginine-9 | RRRRRRRRR | Synthetic Peptide |
| CPP | pVEC | LLIILRRRIRKQAHAHSK | mouse VE cadherin₆₁₅₋₆₃₂ |
| CPP | M918 | MVTVLFRRLRIRRACGPPRVRV | p14ARF₁₋₂₂ |
| CPP | MAP | KLALKLALKALKAALKLA | Synthetic peptide |
| CPP | TP10 | GWTLNSAGYLLGKINLKALAALAKKIL | fusion |
| CPP | FHV | RRRRNRTRRNRRRVR | Flock House Virus |
| | | | Coat Protein₃₅₋₄₉ |
| CPP | PEP-1, | KETWWETWWTEWSQPKKKRKV | Synthetic fusion |
| | Chariot | | |
| CPP | Sweet | (VRLPPP)₃ | Synthetic |
| | Arrow | | |
| | (SAP) | | |
| CPP | Xentry | LCLRPVG | Hep B X-protein |
| Dual | C105Y | CSIPPEVKFNKPFVYLI | α1-anti-trypsin₃₅₉₋₃₇₄ |
| Dual | MPG | GALFLGFLGAAGSTMGAWSQPKSRKV | Synthetic |
| Dual | VP-22 | DAATATRGRSAASRPTERPRAPARSAS | Herpes Simplex Virus |
| | | RPRRPVD | |
| NLS | SV40 | PKKKRKV | Simian Virus 40 |
| | | KRPAATKKAGQAKKKK | |
| NLS | M9 | NQSSNFGPMKGGNFGGRSSGPYGGG | hnRNP A1 |
| | | GQYFAKPRNQGGY | |
| NLS | NRF-2β | EEPPAKRQCIE | Nuclear Respiratory |
| | | | Factor 2 |
| NLS | VpR | DTWTGVEALIRILQQLLFIHFRIGCRHS | HIV VpR |
| | | RIGIIQQRRTRNGA | |

More specifically, the 18 amino acid long pVEC peptide listed in Table 1 is derived from murine vascular endothelial cadherin (VE-cadherin) protein, which mediates physical contact between adjacent cells.

In some embodiments, the method described enables biologically-inert labeling of intracellular compartments in live cells, including cell nuclei. In other embodiments, this approach improves the efficiency of protein transfection into live cells for cell-penetrating fusion proteins described in the art, including recombinant fusions to chemical, pharmaceutical, or fluorescent agents. The method utilizes the observation that a monovalent cell-penetrating peptide can improve cell uptake of an exogenously supplied recombinant fusion peptide when both are supplied separately, or not bound to each other. This enables the formulation of peptide/protein blends at optimal stoichiometries to ensure robust cell uptake and intracellular response utilizing much lower quantities of recombinant protein. In preferred embodiments, the amino acid sequence comprising the cell-penetrating peptide is identical between the monovalent peptide and the recombinant fusion protein. Without describing a particular mode of action, numerous mechanisms are possible to describe how homotypic sequence elements may confer better cell uptake in trans, including homotypic interactions that promote pore-formation as well as multivalent receptor-mediated uptake.

In some embodiments, the present invention utilizes cell-penetrating peptide sequences that do not require multimerization in order to effectively penetrate cells. For example, pVEC and SAP peptides are exemplary cell-penetrating peptides whose multivalency does not significantly improve protein transduction potential in published studies.

In certain embodiments, the cell importation peptide (CPP) employed has at least 85% sequence homology to TAT, Penetratin, Arginine-9, pVEC, M918, MAP, TP10, FHV, PEP-1 (Chariot), Sweet Arrow (SAP), Xentry, or a combination thereof. In preferred embodiments, the CPP is has at least 85% sequence homology to pVEC.

In certain embodiments a construct is formed by a covalent bond between a CPP transporter and a cargo. In certain embodiments, the cargo comprises an imaging agent such as an optical contrast agent, a radioisotopic contrast agent, or a nuclear contrast agent that may provide diagnostic assay when transported into the cell. For example, in certain embodiments the optical contrast agent may be an optical absorber dye, a molecular fluorophores such as, but not limited to, fluorescein, rhodamine, cyanine, coumarin, xanthene, or BIODIPY. In other embodiments the contrast agent may be a SPECT or PET agent. While in still other embodiments the agent may be an isotopically labeled drug or substrate capable of being imaged by NMR spectroscopy.

In certain embodiments the cargo may be a therapeutic agent including biological or molecular therapeutics. Examples of therapeutic agent may be, but not limited to, nucleic acids, signaling peptides, cytotoxic agents or enzyme inhibitors. As such the cargo may result in or confer biological or physicochemical properties to the cell.

In certain embodiments the covalent bond between the CPP and the cargo is an amide such as that formed between an amine and activated ester, a urea formed between an amine and an isocyante, a thioamide, for example formed between an amine and an isothiosyanate, a thioester formed between a thiol and an electrophile (eg. maleimide, alkyl halide), a substituted azobenzenes formed between a phenol and a diazonium salt, a substituted oxime formed between an aldehyde or ketone and a aminoxy compound, a substituted alkylidene hydrazines formed between an aldehyde or ketone and a hydrazino compound, a triazole or substituted triazole formed between an alkyne and an azide, or a thiazolidine heterocycle formed between a terminal cysteine and an aldehyde.

In certain embodiments, a cargo is provided further comprising a NLS segment. As such the NLS segment is bonded to an agent. The NLS-agent is configured to allow the CPP to covalently bond to the NLS, thus forming a CPP-NLS-agent construct. In certain embodiments the NLS segmenta comprises, but are not limited to those listed in aforementioned Table 1; SV40, M9, NRF-2β, or VpR.

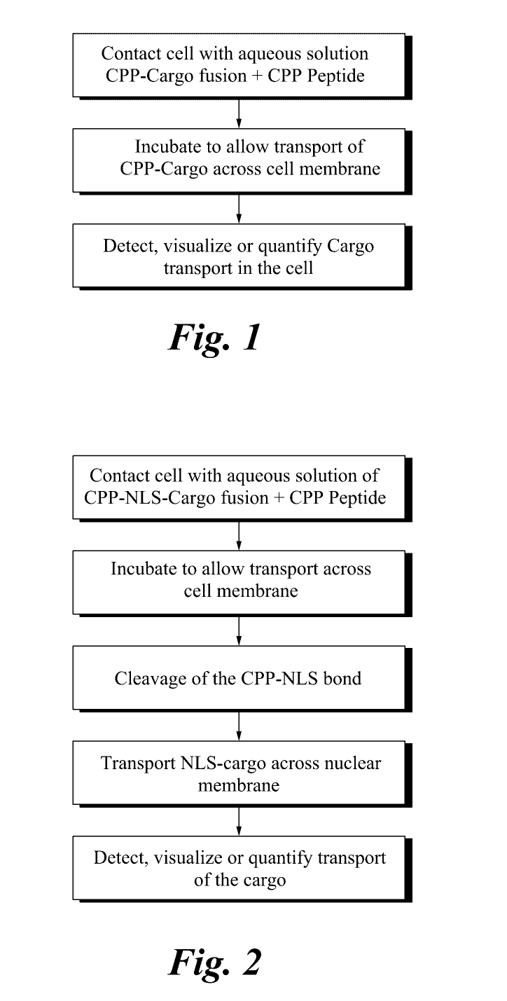

In certain embodiments, a cell penetrating peptide (CPP) is covalently bonded to a cargo to form a construct. An aqueous solution of the CPP-cargo construct and the CPP itself is provided such that the CPP is present both as the functionalized peptide construct as well as the unreactive monomeric form. The aqueous solution is allowed to contact a viable cell allowing for incubation of the cell in the aqueous solution. During incubation, the construct is transported through the cell membrane more efficiently than an aqueous solution containing only the CPP-cargo construct. The presence of the CPP in monomer form results in a beneficial property enhancing the transport of the CPP-construct which may be measured in uptake of the cargo. This is shown in the flowchart in FIG. 1.

In certain embodiments, the aqueous solution may further comprise a pharmaceutical carrier. The pharmaceutical carrier may be used to impart certain properties beneficial to cell transport, solubility, or stabilization of the aqueous solution. As such the carrier may be, but not limited to, alcohol, fats, waxes, co-solvents, buffers, inert solids, or a combination thereof. In certain embodiments the aqueous solution may be a phosphate-buffer saline solution. As such the cell-penetrating transporter for enhancing the delivery of imaging and therapeutic agents to the interior of a cell may comprise a functionalized peptide having a cell importation peptide covalently bound to a cargo, the cell importation peptide in an unreactive monomeric form, and a pharmaceutical carrier.

In certain embodiments, the concentration by weight of the functionalized peptide construct is less than or equal to the unreactive monomeric form of the peptide. In certain embodiments the construct to monomeric peptide is an equal ratio; in still other embodiments the ratio of monomeric peptide to construct may be approximately 3-30 fold and allows for significantly less consumption of the protein construct.

As shown further in FIG. 1, any of a number of detection, visualization, or quantitation techniques, including but not limited to fluorescence microscopy, laser-confocal microscopy, cross-polarization microscopy, autoradiography, magnetic resonance imaging (MRI), magnetic resonance spectroscopy (MRS), or a combination thereof. As such, these, or other applicable methods, or any combination thereof, may be then be used to assess the presence or quantity of the cargo transported across the cell membrane of the tissue sample.

In other embodiments, where the cargo is a therapeutic agent including biological or molecular therapeutics, transporting the cargo across the cellular membrane may result in or confer biological or physicochemical properties to the cell which may be detected as a change in cellular properties, such as metabolism or enzymatic properties or other chemical changes within the cell.

In certain embodiments, the aforementioned covalent bond between the cell importation peptide and the cargo is cleavable. While not necessary in all instances for detection or efficacy in certain embodiments, and the method further comprises releasing the cargo in the interior of the cell.

As shown in the flow chart of FIG. 2, in certain embodiments the CPP-NLS portion of the construct may cleave into two subunits within the reducing environment of cell cytoplasm, thereby releasing a NLS- cargo. The NLS-cargo may then be transported into the cell nucleus. The cargo transported in the nucleus may be an imaging agent, a therapeutic agent or a combination thereof.

As such, in certain embodiments, to allow the cargo-NLS peptide to be transported in the cell nucleus, the construct is formed by a covalent bond cleavable within the cellular membrane. In certain embodiments the covalent bond is a disulfide formed from a thiol and an activated thiol (eg. 3-nitro-2-pyridinesulfenyl cysteine residue (NPyrDS), 4-nitrothiophenol), an ester formed from an alcohol and an activated ester such as an acyl halide, or where the bond is biologically labile, for example from enzymatic cleavage including esterase and protease cleavage.

In certain embodiments the cargo-NLS peptide is constructed such that the cargo is an imaging agent, a therapeutic agent, or a combination thereof. In certain embodiments the cargo is specifically selected to have properties to exhibit nuclear, optical or magnetic contrast; activate or deactivate signaling pathways within the cell; regulate protein synthesis (specifically or generally); induce or suppress apoptosis or mitosis; induce or suppress intra- or inter-cellular signaling; sensitize or desensitize the affected cells towards other therapeutic modalities. Therapeutic modalities may include, but are not limited to, chemotherapeutic, photodynamic, thermal, or ultrasound.

In certain embodiments the use of cell-penetrating transporter as described may further desirable as it allows for more efficient transportation of the cargo across the cellular membrane or nuclear membrane. As such, with certain cargo which may exhibit a toxic or other deleterious effect in the cell, such as but not limited to phototoxicity, the construct and method describes allows for lower concentrations in comparison to standard method.

### EXAMPLES

The following examples are intended only to illustrate methods and embodiments in accordance with the invention, and as such should not be construed as imposing limitations upon the claims. As per industry standards, and appreciated by those skilled in the arts, peptides were prepared using standard laboratory practices. Commercial sources and customized peptide sequences used in the following examples include AnaSpec Inc. (Fremont, Calif.) and Biomatik USA, LLC (Wilmington, Del.).

Results Using pVEC Construct and Monomeric pVEC

A CPP-cargo construct (comprising a cell-penetrating moiety and a nuclear targeting moiety) was synthesized and conjugated via reactive 3-nitro-2-pyridinesulfenyl cysteine residues, and then purified by HPLC. When exogenously supplemented into cell staining media, these peptide fusions are designed to penetrate living cells and cleave into two subunits within the reducing environment of cell cytoplasm, thereby releasing a FITC-NLS peptide that is actively trafficked into the cell nucleus. Thus, the fusion peptide is convenient for discriminating nuclei by live-cell microscopy.

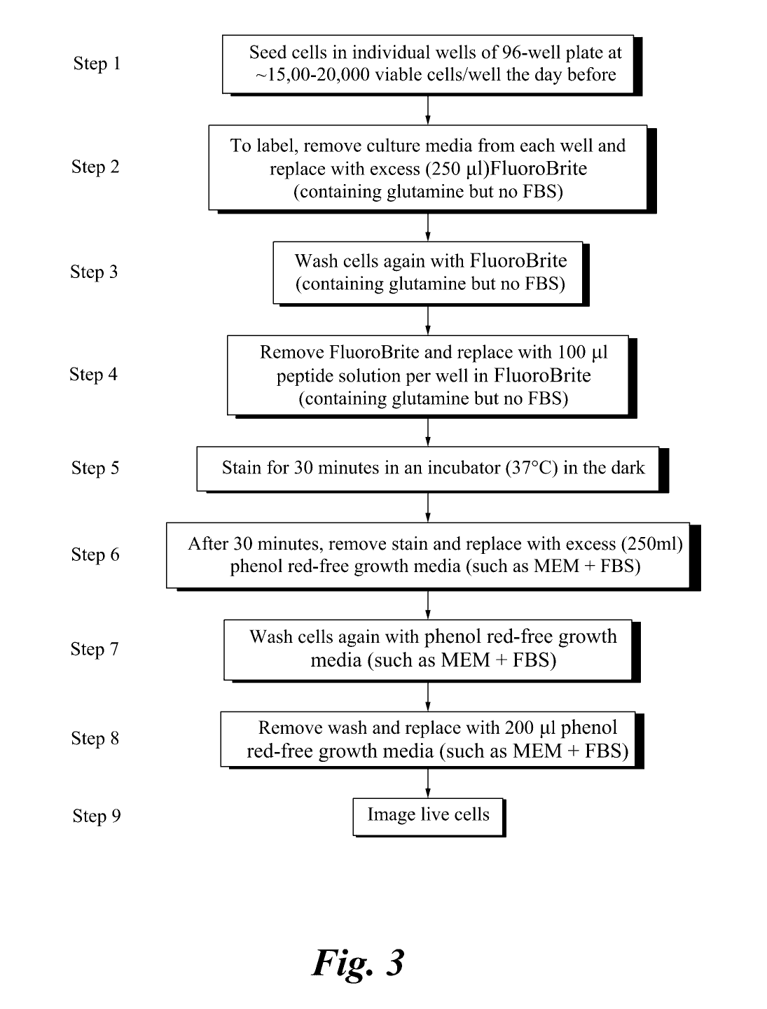

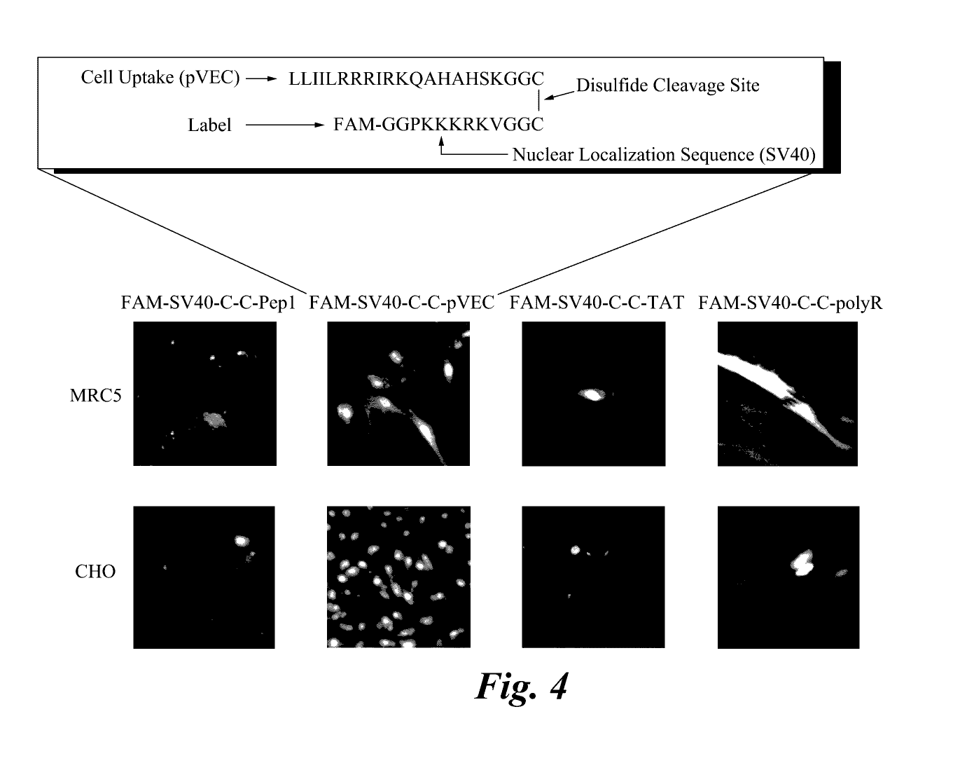

Cell-uptake efficiency for several fusion proteins created in the manner are shown in FIG. 4. Live human MRC5 cells and mammalian CHO cells were incubated with fusion proteins as depicted in the flow chart in FIG. 3, steps 1 through 9. Of the tested cell-penetrating sequences, pVEC demonstrated the highest cell-uptake efficiency and nuclear labeling (FIG. 4). The FITC-NLS/pVEC fusion was constructed by independently synthesizing a FITC-NLS-Npys peptide and a modified pVEC sequence containing a terminal cysteine residue:

Fluorescein-NLS-Npys sequence 1:
5-FAM-GGPKKKRKVGGC(N-Pys)-OH
Modified pVEC sequence 2:
H-LLIILRRRIRKQAHAHSKGGC-OH

These two peptides were fused under acidic conditions to generate a fused peptide disulfide linkage:

Fusion construct sequence 3:
5-FAM-GGPKKKRKVGGCCGGKSHAHAQKRIRRRLIILL

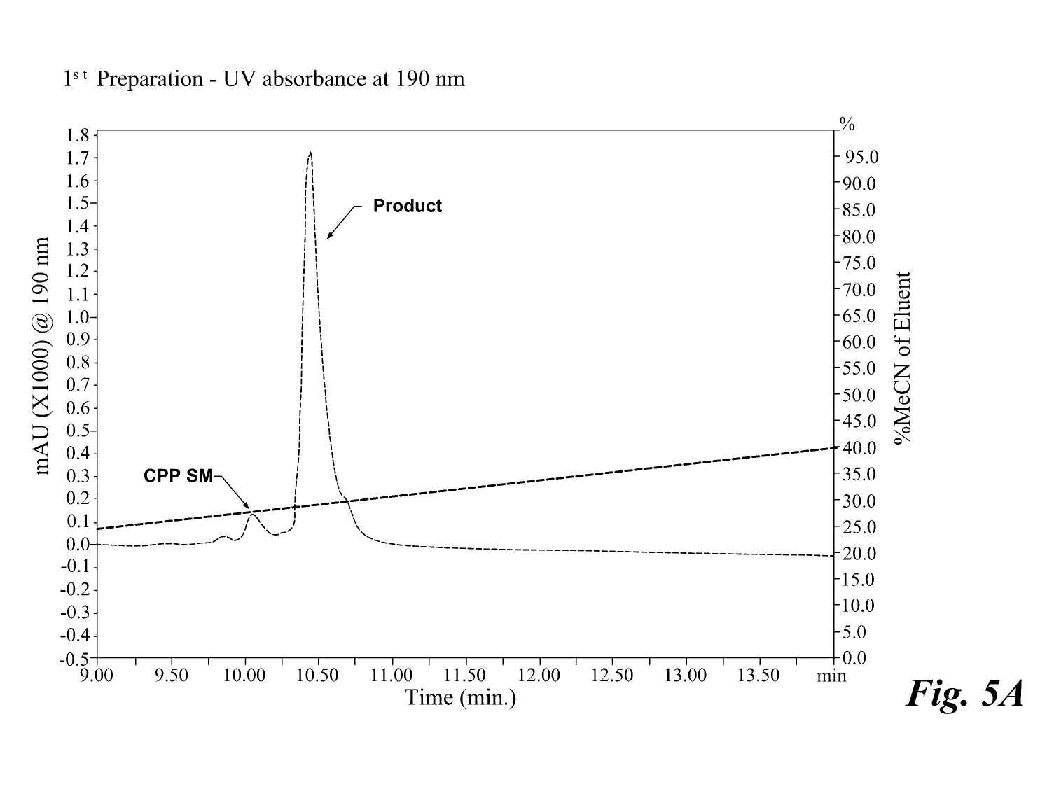

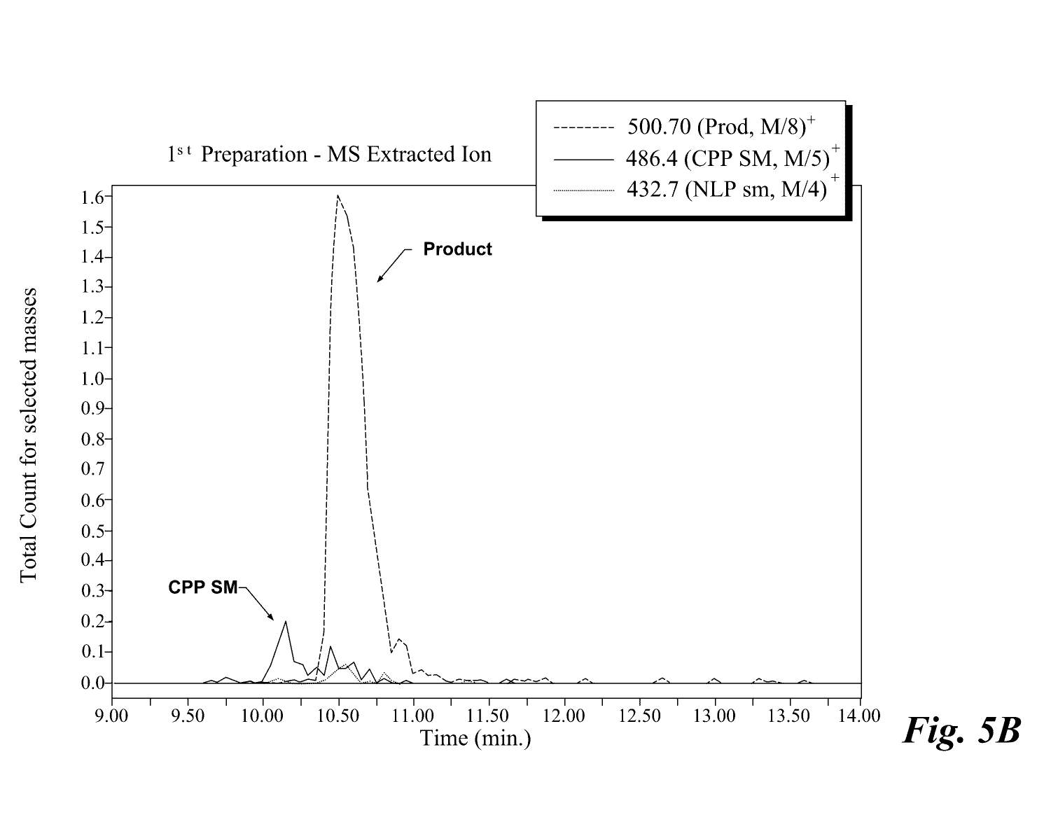

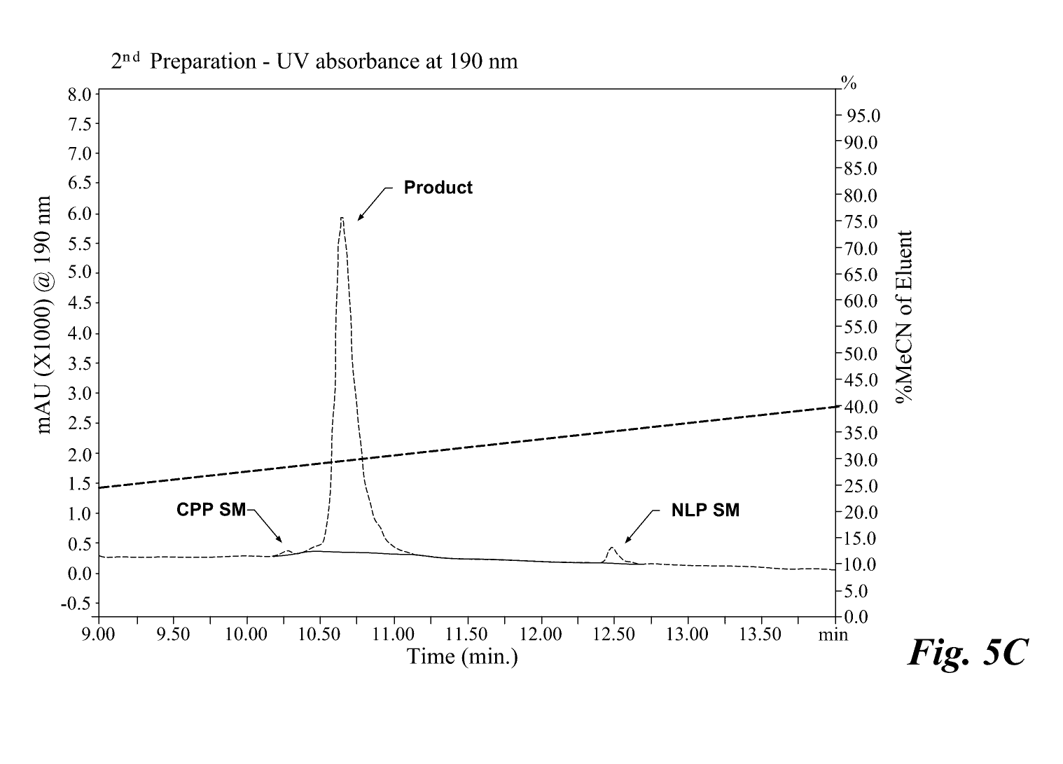

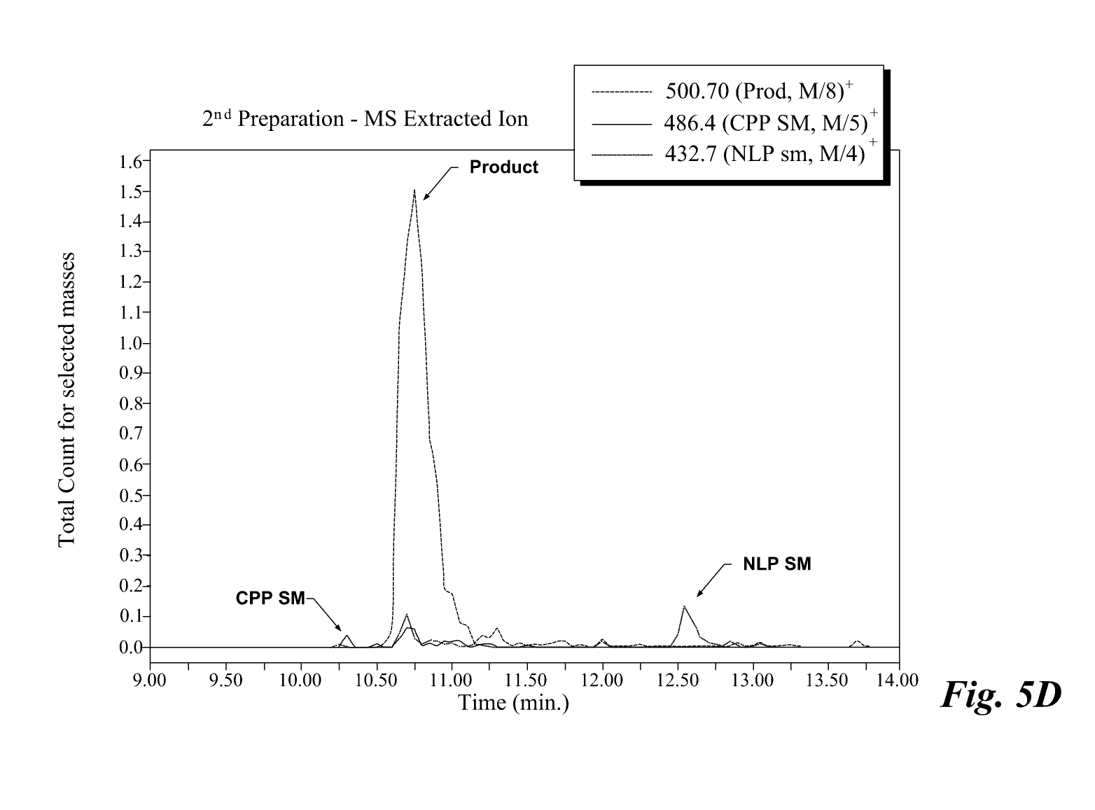

Prior to use for live-cell microscopy, the fusion peptide was purified by HPLC. Subsequent chemical analysis revealed that the first preparation of fusion peptide contained trace quantities of unreacted pVEC peptide (SEQ 1) whereas the second preparation of this fusion peptide was further purified to remove residual unreacted pVEC peptide. FIGS. 5a-d are representative HPLC data comparing multiple batches of fusion protein between pVEC and NLS-FAM. The first preparation was found to contain significant carryover of unconjugated pVEC peptide (CPP SM peak) (FIG. 5a UV absorbance, FIG. 5b, MS) while the second preparation showed only minor carryover of unconjugated NLS-FAM peptide (NLP SM peak) (FIG. 5c UV absorbance, FIG. 5d MS). The HPLC traces further show the purification level of both protein fusions (Product peaks).

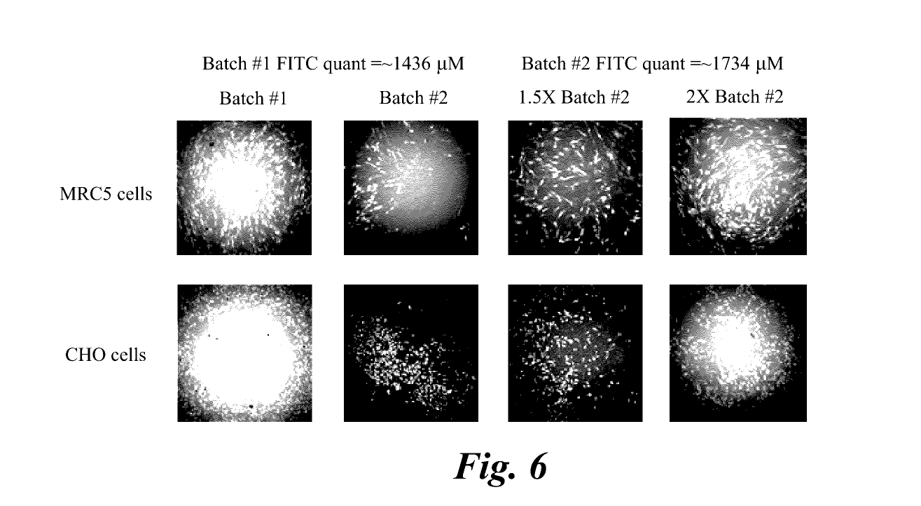

FIG. 6 shows that the first preparation of fusion protein penetrated live cells with greater efficacy than the second preparation. Twice as much fusion protein from the second preparation (which lacked unreacted pVEC) was required to achieve similar cell-labeling as the first preparation of fusion protein (which contained unreacted pVEC). Thus, an aqueous solution having a functionalized protein fusion and an additional quantity of unreactive cell importation peptide in a monomeric form appeared to be beneficial for enhanced cell uptake. As such the presence of a trace amount of the monomeric form, as a residual component, was found to be beneficial component of the solution enabling more efficient transport.

Based on this analytical and live-cell data, we consequently tested cell uptake of a construct containing a fusion peptide (at ˜74 μM or 44 μg/well) in the presence of commercial monovalent pVEC (LLIILRRRIRKQAHAHSK (AnaSpec). For these experiments, the HPLC-purified fusion peptide was resuspended in sterile water to approximately 10 μg/μl and briefly centrifuged at 13,000 rpm for 1 minute to pellet any insoluble material. Then, various amounts of fusion peptide were tested with commercial monomeric pVEC peptide the steps are depicted in the flow chart in FIG. 3, steps 1 through 9.

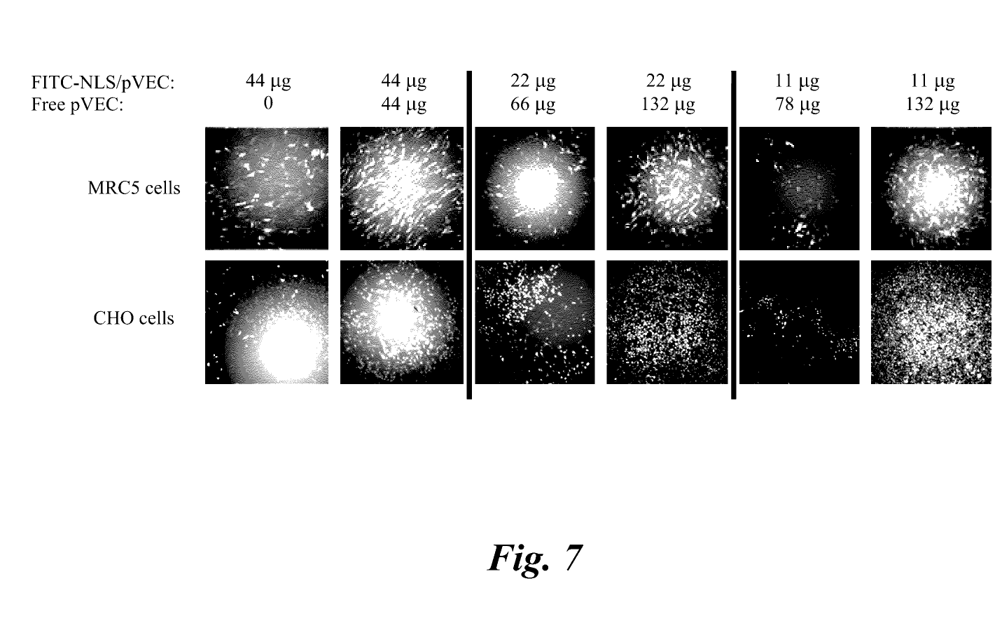

Indeed, FIG. 7 demonstrates enhanced cell uptake and nuclear targeting of FAM-NLS-C-C-pVEC in the presence of excess monovalent pVEC (˜132-156 μg/well), which achieved equal or better cell uptake and nuclear labeling using at least 8-fold less FAM-NLS-C-C-pVEC fusion peptide. In these experiments, explicit addition of monovalent pVEC (post-HPLC purification) validated the efficacy of the monovalent material that was naturally present in the first preparation of FAM-NLS-C-C-pVEC fusion protein depicted in FIGS. 5 and 6.

This finding specifically demonstrates the benefit of having the unreactive CPP peptide in solution. The observation that monovalent pVEC can dramatically enhance the intracellular activity of a fusion peptide also bearing pVEC was not reported in prior findings; multivalency does not significantly improve the protein transduction potential of pVEC when covalently linked (Eggimann, G. A., et al., Convergent synthesis and cellular uptake of multivalent cell penetrating peptides derived from Tat, Antp, pVEC, TP10 and SAP. Organic & Biomolecular Chemistry, 2013. 11(39): p. 6717-6733).

To investigate if monovalent pVEC might also function with heterologous peptides, a set of control experiments were conducted in which live cells were incubated with pVEC and heterologous fusion proteins (FAM-NLS-C-C-Pepl or FAM-NLS-C-C-TAT1), or with control peptide (FAM-NLS-Npys; 5-FAM-GGPKKKRKVGGC (N-pys)-OH). Heterologous FITC-NLS/CPP fusions were constructed by independently synthesizing FITC-NLS-Npys and two additional modified PEP-1 or TAT1 sequences containing terminal cysteine residues:

FITC-NLS-Npys sequence 1:
5-FAM-GGPKKKRKVGGC(N-Pys)-OH
Modified pVEC sequence 2:
H-LLIILRRRIRKQAHAHSKGGC-OH
Modified PEP-1 sequence 4:
H-CGGKETWWETWWTEWSQPKKKRKV-OH
Modified TAT1 sequence 5:
H-GRKKRRQRRRPPQC-OH

These peptide domains were fused under acidic conditions to generate fused peptide disulfide linkages: FITC-NLS/PEP-1 fusion sequence 6:

5-FAM-GGPKKKRKVGGCCGGKETWWETWWTEWSQPKKKRKV
FITC-NLS/TAT construct sequence 7:
5-FAM-GGPKKKRKVGGCCQPPRRRQRRKKRG

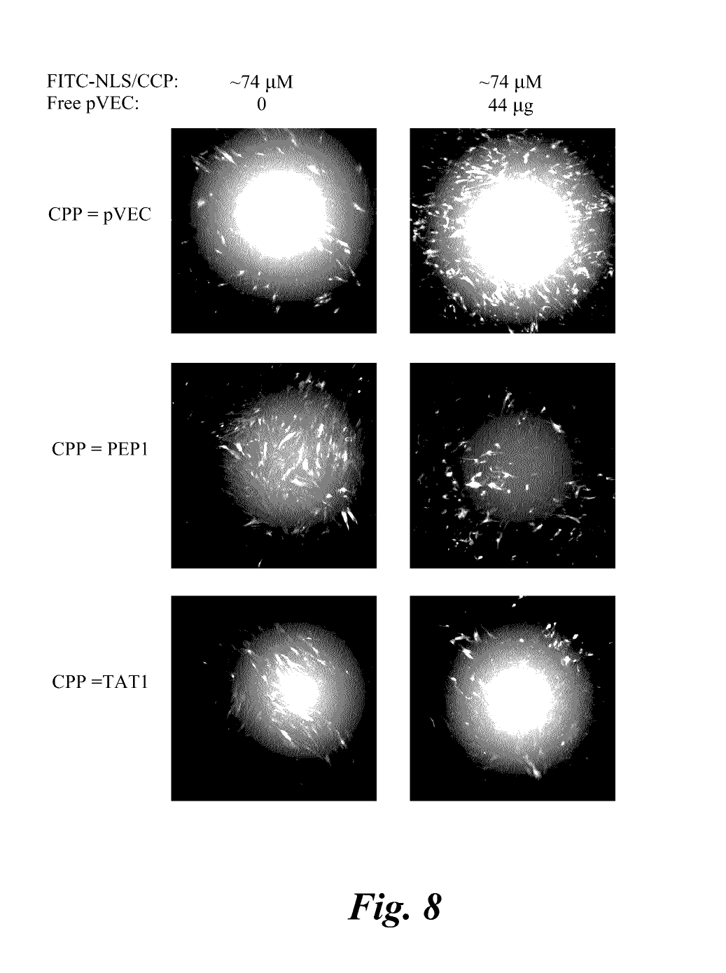

FIG. 8 illustrates that monovalent pVEC (˜44 μg/well) only enhanced the cellular uptake of homologous FAM-NLS-C-C-pVEC fusion peptide in MRC5 cells, unlike either heterologous PEP1 or TAT1 fusions.

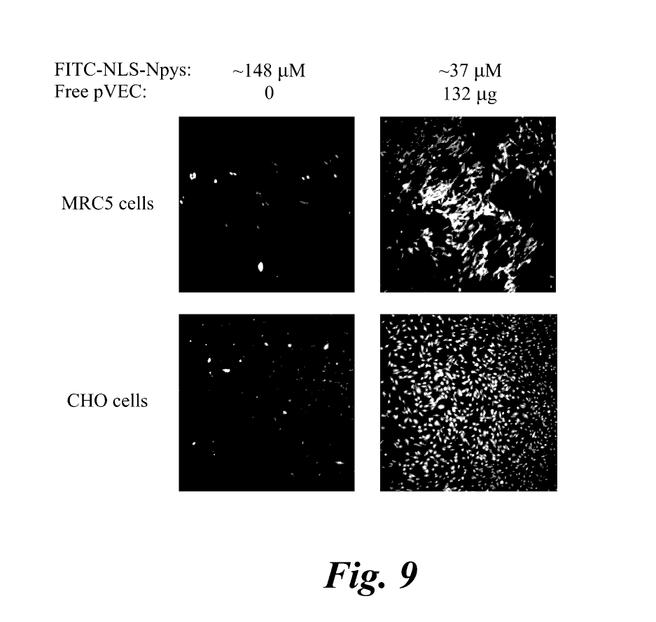

Using higher amounts of monovalent pVEC (˜132 μg/well), enhanced cell uptake of control peptide (FITC-NLS-Npys) was also observed, possibly suggesting that pVEC may exhibit both homologous and heterologous specificity depending on the concentration of monovalent pVEC applied to cells. This is shown further in FIG. 9. However, a portion of this FITC-NLS-Npys control peptide may have successfully conjugated with monovalent pVEC at reactive lysine residues (in culture media without preparative HPLC), thereby creating a non-cleavable FITC-NLS-pVEC fusion during the cell staining step. In this scenario, any unreacted monovalent pVEC may enhance cell uptake of the non-cleavable FITC-NLS-pVEC fusion peptide through the homologous mechanism demonstrated in FIG. 8.

While only certain features of the invention have been illustrated and described herein, many modifications and changes will occur to those skilled in the art. It is, therefore, to be understood that the appended claims are intended to cover all such modifications and changes as fall within the true spirit of the invention.

What is claimed is:

1. A cell-penetrating transporter for enhancing the delivery of imaging and therapeutic agents to the interior of a cell, the agent comprising:
a functionalized peptide construct comprising a cell importation peptide covalently bound to a cargo;
the cell importation peptide in an unreactive monomeric form; and
a pharmaceutical carrier.

2. The transporter of claim 1 wherein the cargo is an imaging agent, a therapeutic agent, or a combination thereof.

3. The transporter of claim 1, where the cell importation peptide has at least 85% sequence homology to TAT, Penetratin, Arginine-9, pVEC, M918, MAP, TP10, FHV, PEP-1 (Chariot), Sweet Arrow (SAP), Xentry, or a combination thereof.

4. The transporter of claim 3 where the cell importation peptide has at least 85% sequence homology to pVEC.

5. The transporter of claim 1 where the covalent bond between the cell importation peptide and the cargo is is an amide, urea, thioamide, thioester, substituted azobenzene, substituted oxime, substituted alkylindene hydrazine, thiazole, substituted triazole, thiazolidine heterocycle, or a combination thereof.

6. The transporter of claim 1 where the covalent bond between the cell importation peptide and the cargo is cleavable and the method further comprises releasing the cargo in the interior of the cell.

7. The transporter of claim 6 where the cleavable bond is a disulfide, an ester, biologically labile, or a combination thereof.

8. The transporter of claim 7 where the cleavable bond comprises a disulfide.

9. The transporter of claim 7 where the biologically labile bond is cleavable by an enzymatic reaction.

10. The transporter of claim 6, where the cargo comprises a nuclear localization sequences (NLS) bonded to at least one of an imaging agent or a therapeutic agent.

11. The transporter of claim of claim 10 where the NLS peptide has a at least 85% sequence homology to amino acids 126-132 of simian virus large T antigen importin binding sequence (SV40).

12. The transporter of claim 11 wherein the NLS has at least 85% sequence homology to X. laevis nucleoplasmin amino acids 164-172 (M9 peptide).

13. The transporter of claim 1 where the pharmaceutical carrier is an aqueous solution.

14. The transporter of claim 13 where the aqueous solution is a phosphate-buffer saline solution.

15. The transporter of claim 1 where the concentration by weight of the functionalized peptide construct is less than or equal to the unreactive monomeric form of the peptide.
